(19) United States
(12) Patent Application Publication
(10) Pub. No.: US 2007/0238972 A1
(43) Pub. Date: Oct. 11, 2007

(54) Method for generating MR (magnetic resonance) images of a moving partial area of an object

(76) Inventor: Arno Nauerth, Erlenbach (DE)

Correspondence Address: KOHLER SCHMID MOEBUS, RUPPMANNSTRASSE 27, D-70565 STUTTGART, DE

(21) Appl. No.: 11/654,540

(22) Filed: Jan. 18, 2007

(30) Foreign Application Priority Data
Jan. 21, 2006 (DE) 10 2006 002 982.8

(51) Int. Cl. A61B 5/05

(52) U.S. Cl. 600/410; 600/420

(57) ABSTRACT

A method for generating MR (magnetic resonance) images of a moving partial area of an object with a repeating motion sequence over comparable motion states, wherein an MR data set, which is encoded for generating an individual MR image of the object, is provided for each motion state from a plurality of successive individual MR measurements with shorter time intervals than a repetition rate of the motion sequence, and wherein at least one navigator data point is generated for each individual MR measurement as an indicator for the comparability of several motion states, is characterized in that a position of the partial area is determined for each individual MR image, from which a function f(t) of the time shift of the position is determined, and the measuring data of the individual MR measurement is phase-corrected in correspondence with its respective motion state using the function f(t) to keep the position of the partial area in a spatially stationary state. This permits observation of a moving partial area irrespective of its motion state, such that rapid changes within the partial area can be observed.

The invention concerns a method for generating MR (magnetic resonance) images of a moving partial area of an object with at least one repeating motion sequence through comparable motion states, wherein an MR data set, which is encoded for generating an individual MR image of the object, is provided for each motion state from a plurality of successive individual MR measurements with shorter time intervals than a repetition rate of the motion sequence, and wherein at least one navigator data point is generated for each individual MR measurement as an indicator for the comparability of several motion states.

Methods of this type for imaging moving objects are disclosed in Journal of Magnetic Resonance in Medicine, MRM 13 (2005).

The acquisition of high-resolution MR images of moving objects is required, in particular, in clinical applications. These are mainly images of the heart, lungs or the abdominal area, wherein the heart beat and breathing motion produce a relative motion between the object to be imaged and the magnetic field generated by the MR apparatus. Acquisition of a 2- or 3-dimensional image requires repeated application of imaging pulse sequences with different phase encoding gradients and reconstruction of the image. The individual MR signals are acquired in different motion states due to a continuous motion sequence of the object, which comprises in most cases a plurality of motion states. This can produce artefacts, such as ghosts, distortions and deterioration of the resolution in the reconstructed image.

This problem is conventionally solved by triggering the times of the individual measurements at comparable motion states of the object using external sensors. This requires, however, more time, in particular, when several individual motions with different frequencies overlap. In this method, the user must always wait for a certain flank of the trigger signal which characterizes a certain motion state of the object, and for this reason, the spin system may be in different relaxation states, leading to different signal strengths.

Moreover, individual MR measurements are conventionally associated with certain motion states using navigator signals. In an investigation of a change with time of a partial area of the object to be investigated, the time intervals between two identical motion states are often too large to observe the desired effects.

U.S. Pat. No. 6,552,541 B2 describes a method for correcting disturbing influences on the MR signals of a substantially stationary sample which is disposed in the measuring volume of an MR apparatus, wherein an RF excitation pulse is irradiated onto the substance and a time-dependent MR signal, generated thereby, is detected and digitized in a phase-sensitive manner. A time dependence of the phase of the MR signal is thereby determined from a time dependence of the detected and digitized MR signal with reference to a predetermined reference phase of a reference signal, and is digitized, and one or more correction or control values are determined therefrom. In this method, almost any measuring point is used for determining the magnetic field shift, and thereby for the control. This ensures great control accuracy.

It is the underlying purpose of the invention to propose a method of the above-described type which permits observation of the moving partial area of the object irrespective of its motion state, such that both observation of rapid changes within the partial area and observation over a longer period are possible.

This object is achieved in accordance with the invention in that a one-, two- or three-dimensional position of the partial area is determined for each individual MR image, from which a function f(t) of the time shift of the position is determined, and the measured data of the individual MR measurement are phase-corrected in correspondence with their respective motion state using the function f(t) to keep the position of the partial area in a spatially stationary state.

Phase correction causes a shift of the coordinates of the corresponding image points. After phase correction of the individual MR measurements, these can be combined into MR images. These MR images comprise directly successive individual MR measurements irrespective of the motion states in which these individual measurements were acquired.

In this fashion, disturbing motion sequences can be compensated in each individual MR measurement, such that acquisitions of the previously fixed interesting partial area of the object can be observed without being influenced by disturbing motion sequences. In contrast to conventional methods, the inventive method permits direct comparison between two individual MR images acquired in different motion phases, wherein the partial area is kept stationary. In this fashion, all acquired individual MR measurements can be used for further evaluation.

With particular advantage, a film of the partial area is produced as a function of time, in particular, comprising a time scale which is much larger than the time scale of the motion sequence. Changes of the partial area may thereby be observed over a long period of time without being influenced by disturbing motion sequences. In the inventive method, each individual MR measurement can, in principle, be used for data evaluation irrespective of the motion state in which it was acquired. Acquisitions over a longer period of time thereby have considerably improved image quality due to increased accumulation of individual MR measurements.

This is of interest e.g. when the individual MR measurements of the film are acquired during and/or after application of an active substance, in particular, a contrast medium.

The inventive method is particularly advantageous for producing a film of a further, preferably non-periodic, motion sequence, e.g. the peristalsis. The inventive correction permits acquisition and analysis of the non-periodic motion sequence which is isolated from the other motion sequences.

In a preferred variant, the method is performed for several different motion sequences which overlap with time, in particular, for heartbeat and breathing of a living object.

The inventive method is preferably a nuclear magnetic resonance (NMR) method.

With particular preference, at least one coherent area of successive data points within the individual MR measurement is used as an indicator, wherein this coherent area is identically repeated with respect to irradiated RF (radio frequency) pulses and switched gradients for all individual MR measurements within the respective MR measuring sequence. In this fashion, additional information can be obtained from an MR experiment for determining the motion states of the motion sequence. In this fashion, the individual measurements can be associated with the corresponding motion states with high accuracy using this indicator. This eliminates or at least greatly reduces artefacts in the resulting MR images.

In an advantageous further development of this variant, the coherent area comprises a partial area, in which a rephasing gradient, in particular, a slice selection gradient is applied, thereby rephasing the nuclear spin system in this partial area. The measuring time for the indicator may then be reduced, thereby still obtaining an acceptable signal strength in view of the rephasing process.

With particular advantage, further individual MR measurements are performed with a second type of nucleus at the same time as the individual MR measurements of a first type of nucleus to determine the indicator. The indicator may thereby be measured with high temporal resolution without disturbing the actual data acquisition. The slice thickness that provides the indicator signal may moreover have almost any thickness to optimize the signal amplitude of the indicator, while the individual measurements can be performed with thin slices to obtain high spatial resolution.

The inventive method produces MR images in different motion phases, which can be represented in the form of a film without showing the influence of disturbing motion sequences. The MR images of a complete breathing and/or heart cycle of a living object to be examined may e.g. be used to represent the partial area of interest, such that changes within the partial area that take place within one motion cycle can be imaged or observed without being falsified by this motion cycle. The partial area of interest is thereby kept in a quasi stationary state to provide a meaningful direct comparison of the partial area. It is thereby e.g. possible to produce MR images of a complete breathing and/or heart cycle of an individual to be examined from different MR images that are associated with different motion states.

Further advantages of the invention can be extracted from the following detailed description. The features mentioned above and below may be used individually or collectively in arbitrary combination. The variants disclosed and described are not to be understood as exhaustive enumeration but have exemplary character for describing the invention.

The starting point of the inventive method is the acquisition of individual MR measurements of a partial area which is moved by external circumstances which are not of interest, e.g. breathing of the patient under examination. The imaged investigation volume should thereby be selected such that the motion of the partial area takes place within the volume under investigation. The motion state for each individual MR measurement is now determined through navigator measurement. The individual MR measurements of one motion state are then combined into one MR image. Such MR images now comprise individual MR measurements which were partly acquired within long time intervals which prevents observation of rapid changes within the partial area.

Through a comparison of such MR images from different motion phases of the object under investigation, in particular, of individual images of a film, the position of the partial area of interest may be determined manually or through pattern recognition, and the time shift of the position of this partial area can be described by a function f(t). With this function f(t), all individual MR measurements of the MR images can be phase-corrected, such that the position of the partial area is kept in a spatially stationary state. A corresponding phase shift is thereby performed for each echo point of the individual MR measurements. The measuring data is thereby not corrected in the produced MR images, rather in the original individual MR measurements. These corrected individual MR measurements can be combined into new MR images such that, if desired, a film of the partial area can be produced, which contains all individual MR measurements in chronological order, thereby still keeping the position of the partial area in a stationary state. In contrast to prior art, fast changes within the partial area can thereby be observed with great accuracy. In general, several individual MR measurements are combined to improve the signal-to-noise ratio.

The inventive method also permits corresponding correction of several overlapping periodic motion sequences. When, e.g. an artery (partial area of interest) of a living object shall be observed in order to control the concentration development of an administered contrast medium, this artery will move within certain limits due to breathing and also due to the heart beat of the object under investigation. The motions caused by heartbeat and breathing of the examined object, which are initially superposed with a motion sequence, can be separated using a data processing program and using certain predetermined parameters. In this fashion, the two motion sequences can be corrected independently of each other.

This method also permits separation of individual motion sequences which are superposed by periodic motion sequences. The peristalsis can be observed e.g. irrespective of the patient's breathing.

In any case, the inventive method permits individual correction of each individual MR measurement and thereby production of a large number of MR images within a minimum time pattern. This realizes an extremely high time resolution for the MR acquisitions. Moreover, the time required for the necessary MR measurements can be greatly reduced, since each individual MR measurement can now be used for evaluation. SPECIFICATION AMENDMENTS: On page one, insert as a title prior to the first paragraph - BACKGROUND OF THE INVENTION -. On page 3, insert as a title prior to the first full paragraph 0. SUMMARY OF THE INVENTION -. On page 6, insert as a title prior to the second full paragraph -. DESCRIPTION OF THE PREFERRED EMBODIMENT -. On page 8 line one, replace “Claims” with - I CLAIM: -.

1-9. (canceled)

10. A method for generating MR (magnetic resonance) images of a moving partial area of an object, the partial area exercising a repeating motion sequence through comparable motion states, the method comprising the steps of:
a) recording a plurality of successive individual MR measurements in time intervals which are shorter than a repetition rate of the motion sequence;
b) generating at least one navigator data point for each individual MR measurement as an indicator of comparability of several motion states;
c) determining a one, two, or three dimensional position of the partial area for each individual MR image;
d) determining a function f(t) of a time shift in the positions of step c);
e) phase correcting measuring data of the individual MR measurements in correspondence with respective motion states thereof using the function f(t) of step d) to keep positions of the partial area in a substantially stationary state; and
f) generating an MR image of the object for each motion state.

11. The method of claim 10, wherein a film of the partial area is produced as a function of time.

12. The method of claim 11, wherein the film has a time scale which is much larger than a time scale of the motion sequence.

13. The method of claim 11, wherein individual MR measurements of the film are acquired during and/or after administering an active substance or a contrast medium.

14. The method of claim 11, wherein the film depicts a non-periodic motion sequence or a peristalsis.

15. The method of claim 10, wherein the method is performed for several different motion sequences which overlap in time.

16. The method of claim 15, wherein the several different motion sequences comprise a heart beat and a breathing of a living entity.

17. The method of claim 10, wherein the method is a nuclear magnetic resonance (NMR) method.

18. The method of claim 10, wherein the indicator of step b) comprises at least one coherent area of successive data points within the individual MR measurement, this coherent area being identically repeated with respect to irradiated RF (radio frequency) pulses and switched gradients for all individual MR measurements within a respective MR measuring sequence.

19. The method of claim 18, wherein the method is a nuclear magnetic resonance method (NMR), the coherent area comprising a partial area, in which a rephasing gradient or a slice selection gradient is applied, thereby rephasing a nuclear spin system in this partial area.

20. The method of claim 17, wherein further individual MR measurements are performed with a second type of nucleus simultaneously with individual MR measurements of a first type of nucleus to determine the indicator.
